(19) United States
(12) Patent Application Publication
(10) Pub. No.: US 2025/0345475 A1
(43) Pub. Date: Nov. 13, 2025

(54) APPARATUS AND METHOD FOR TREATING AND IN PARTICULAR STERILIZING PLASTIC PREFORMS OR CONTAINERS

(71) Applicant: KRONES AG, Neutraubling (DE)

(72) Inventors: Juergen SOELLNER, Beratzhausen (DE); Florian GELTINGER, Donaustauf (DE); Max BRIKMANN, Thalmassing (DE)

(21) Appl. No.: 19/201,507

(22) Filed: May 7, 2025

(30) Foreign Application Priority Data
May 7, 2024 (DE) 10 2024 112 824.0

(51) Int. Cl. A61L 2/20; A61L 2/24

(52) U.S. Cl. CPC A61L 2/208; A61L 2/24; A61L 2202/11; A61L 2202/122; A61L 2202/14; A61L 2202/15; A61L 2202/23

(57) ABSTRACT

Apparatus for treating and in particular sterilizing plastic preforms and/or containers, including a transport device configured for transporting the plastic preforms during their treatment, a housing forming a treatment chamber within which the transport device is arranged, a generating device for generating a flowable treatment agent, and a supply device configured for supplying the sterilization agent generated by the generating device to the treatment chamber. The apparatus has a first discharge device configured for removing a flowable medium enriched with the sterilization agent from the treatment chamber, and a return device configured for returning at least part of the medium removed from the treatment chamber back to this treatment chamber.

### CROSS REFERENCE TO RELATED APPLICATION

This application claims priority to German Patent Application Serial No. 10 2024 112 824.0, filed May 7, 2024, the contents of which are incorporated herein by reference.

### BACKGROUND OF THE INVENTION

The present invention relates to an apparatus and to a method for treating and in particular sterilizing plastic preforms or containers. It has long been known in the prior art to sterilize containers or plastic preforms, for example, before certain treatment processes such as a filling process.

In the case of plastic preforms, it has recently become known to sterilize them and then to form them into plastic bottles using a sterile blow molding machine. Different procedures are known for the purpose of this sterilization. For example, it is known to sterilize containers and/or plastic preforms by UV radiation or X-rays or electron beams.

However, it has long been known in the prior art to carry out sterilization using flowable sterilization agents such as hydrogen peroxide or peracetic acid. For this purpose, the plastic preforms or containers are usually applied with a flowable sterilization agent within a treatment chamber.

The problem in this case is that the consumption of sterilizing agents is comparatively high.

The present invention is therefore based on the object of reducing the consumption of sterilization agents.

### SUMMARY OF THE INVENTION

An apparatus according to the invention for treating and in particular cleaning and/or sterilizing plastic preforms and/or containers has a transport device which is suitable and intended to transport the plastic preforms and/or containers at least temporarily during their treatment. Preferably, these plastic preforms (and/or containers) are transported at least temporarily and preferably permanently during their treatment. Preferably, these plastic preforms (and/or containers) are transported continuously and/or the transport device is suitable and intended to transport the plastic preforms (and/or containers) continuously during their treatment.

Furthermore, the apparatus has a housing forming a treatment chamber, within which the transport device is arranged. Particularly preferably, this housing surrounds the treatment chamber substantially completely (with the exception of any airlocks through which plastic preforms and/or containers can be supplied into the treatment chamber or removed from the treatment chamber). Particularly preferably, the housing closes off the treatment chamber in a substantially gas-tight manner (again with the exception of any airlocks which serve for the supply or discharge of plastic preforms or containers).

Furthermore, the apparatus comprises a generating device for generating a flowable treatment agent and/or a reservoir for storing a flowable treatment agent. In particular, the treatment agent is a sterilization agent.

Furthermore, a supply device is provided which is suitable and intended to supply the treatment agent produced by the generating device and in particular the sterilization agent, and/or the sterilization agent stored in the storage means, to the treatment chamber.

According to the invention, the apparatus comprises a first discharge device which is suitable and intended to remove a flowable medium and in particular a flowable medium enriched with a sterilization agent from the treatment chamber, and a return device which is suitable and intended to return at least part of the medium removed from the treatment chamber or of the flowable medium back into the treatment chamber and/or a further treatment chamber.

Preferably, the flowable medium is removed from a first treatment chamber and supplied to a second treatment chamber via a return device, wherein the second treatment chamber is different from the first treatment chamber. The treatment chambers can be of the same type and/or different types, such as a filler, closure, etc., preferably filler or closure treatment chambers.

Preferably, the flowable medium is removed from either—in particular, the first—treatment chamber and supplied via a return device to any number of further treatment chambers, preferably individually, preferably in parts to each further treatment chamber.

As mentioned, the problem in the prior art is that efficient disinfection requires a high flow velocity and thus a large amount of H₂O₂. In the prior art, this amount is provided entirely from an evaporator or the mentioned generating device, which leads to a high consumption of H₂O₂ gas.

Within the scope of the invention, it is therefore proposed to remove the gas originating from the treatment of the plastic preforms and/or the treatment chamber and to supply it and/or mix it into a fresh gas stream, in particular via a compressor. Preferably, as described in more detail below, the circulation volume is controlled via pressure gauges, flow meters and/or frequency converters. The frequency converter can be used to control the speed of the compressor and thus adjust the pressure or volume flow to a specified value.

In this way, cost savings of up to 66% can be achieved for a machine operator. In addition, it can lead to a reduction in the consumption of particularly problematic and/or harmful chemicals. In addition, it is also possible to seal off the treatment chamber or the gas supply from the surroundings much more easily.

Particularly preferably, the generating device is an evaporator. The sterilization agent is particularly preferably hydrogen peroxide (H₂O₂) gas.

In a further preferred embodiment, the apparatus comprises at least one mixing device for mixing at least a part of the flowable medium removed from the treatment chamber with the flowable medium generated by the generating device. In the simplest case, this mixing device can be a mixing chamber into which the two components can be supplied.

In particular, a mixing ratio between these two media can be set and/or controlled.

Particularly preferably, this mixing device is arranged outside the treatment chamber. It is thus possible for this mixing device to have a plurality of valves by which the mixing ratio between the medium taken from the treatment chamber and the medium (freshly) produced by the generating device can be determined.

For example, it is possible to combine two or more flow lines for mixing purposes. In addition, the mixing device can have a mixing chamber in which on the one hand a mixture of the removed medium and on the other hand the produced or generated medium is achieved.

In a further advantageous embodiment, the apparatus has a control device which is suitable and intended to control and/or regulate a mixing ratio of the medium to be supplied to the treatment chamber.

In particular, it is possible to control the proportions of the medium produced by the generating device on the one hand and the medium taken from the treatment chamber on the other hand.

For example, the mixing device can be controlled so that the sterilization medium to be supplied to the treatment chamber consists of 40% freshly produced sterilization medium and 60% medium taken from the treatment chamber.

Several approaches are conceivable to control and/or regulate this mixing ratio. It is conceivable that a pump output with which the flowable medium is removed from the treatment chamber is controlled and/or regulated. The mixing ratio can also be regulated with a plurality of valves arranged in the respective supply lines for the medium taken from the treatment chamber and the medium produced by the generating device.

In addition, it is possible to provide a mixing valve in the mixing device and/or a mixing area which controls or regulates the proportions.

In a further advantageous embodiment, the apparatus has a second discharge device which is suitable and intended to remove a flowable medium and in particular a flowable medium enriched with the sterilization agent from the treatment chamber, wherein this second discharge device is preferably spaced apart from the first discharge device.

For example, the at least two discharge devices can be arranged on opposite walls of the above-mentioned housing. Particularly preferably, the transport device is located at least partially between the at least two discharge devices. In this way, it is possible to remove or discharge the flowable medium from several areas of the housing and/or the treatment chamber, which in particular enables uniform removal or discharge.

Preferably, a return device is also provided which is suitable and intended to return at least a part of the medium removed from the treatment chamber by the second discharge device back into this treatment chamber. This return device is preferably suitable and intended to return the medium discharged from the treatment chamber to the treatment chamber, wherein this supply preferably takes place after this medium has been mixed with freshly produced sterilization medium.

It is possible that the fluid streams of the two discharge devices are first combined, and are then combined with the stream of the freshly produced sterilization agent. However, it would also be conceivable that the fluid streams of the discharge devices are supplied one after the other to the stream of the freshly produced sterilization agent. It would also be conceivable that the fluid streams from the two discharge devices are supplied together with the freshly produced sterilization agent into a common mixing chamber.

In a further preferred embodiment, the mixing device is suitable and intended to mix the medium removed from both return devices with the freshly produced sterilization medium.

In a further advantageous embodiment, the apparatus has at least one application device arranged within the treatment chamber to apply the flowable medium and in particular the flowable sterilization agent to the plastic preforms and/or containers transported by the transport device. In particular, this application device is suitable and intended to apply the already mixed substance to the plastic preforms.

Particularly preferably, the application device comprises movable application elements and in particular application elements which are moved together with the plastic preforms to be sterilized.

Particularly preferably, the application device comprises a line system which preferably comprises a plurality of treatment nozzles which apply the sterilization agent to the plastic preforms.

In a further advantageous embodiment, a distribution device, in particular a rotary distributor, is also provided in order to distribute the flowable medium and in particular the sterilization agent (in particular within the treatment chamber) to a plurality of application devices and in particular application nozzles arranged on a transport carrier and/or sterilization wheel.

In a further preferred embodiment, the transport device has at least one first transport carrier rotatable about a specified axis of rotation, on which a plurality of holding devices for holding the plastic preforms or containers is arranged. Preferably, these holding devices are arranged equidistantly on the rotatable transport carrier. Particularly preferably, these holding devices are arranged in such a way that the plastic preforms are transported one after the other. Preferably, the holding devices are arranged such that successive plastic preforms held by these holding devices have a distance from one another which is greater than 2 cm, preferably greater than 3 cm and preferably greater than 4 cm.

Preferably, the holding devices are arranged such that successive plastic preforms held by these holding devices have a distance from one another which is less than 20 cm, preferably less than 18 cm, preferably less than 15 cm, preferably less than 12 cm and particularly preferably less than 10 cm.

Particularly preferably, these holding devices are resistant to the sterilization agent. Particularly preferably, the holding devices are gripping devices which are suitable and intended to grip the plastic preforms or containers in the region of their mouths and in particular below a support ring of the plastic preforms or containers.

In a further preferred embodiment, a supply device is provided which supplies the plastic preforms or containers to the holding devices of the transport device. Preferably, this supply device is also arranged within the treatment chamber. Preferably, this supply device also has application devices for applying flowable sterilization agent to the plastic preforms or containers. Preferably, at least three and preferably exactly three transport wheels equipped with holding devices are provided within the treatment chamber, and the plastic preforms are each transported by all of these transport wheels or can be transported one after the other by all of these transport wheels.

Particularly preferably, the apparatus also comprises a discharge device which is suitable and intended to discharge the plastic preforms or containers from the transport device. Preferably, this discharge device is also arranged within the treatment chamber. Preferably, this discharge device also has application devices for applying the flowable sterilization agent to the plastic preforms or containers. Preferably, this discharge device is a component of the transport device.

In a further advantageous embodiment, the supply device also has a rotatable carrier on which a plurality of the holding devices are arranged. In a further advantageous embodiment, the discharge device also has a rotatable carrier on which a plurality of the holding devices are arranged. Particularly preferably, an axis of rotation of the supply device and/or the discharge device is arranged parallel to the axis of rotation of the transport device.

Particularly preferably, at least three such rotatable carriers and preferably exactly three such carriers are arranged within the treatment chamber.

In a further preferred embodiment, the apparatus has at least one sensor device which is suitable and intended to detect at least one measured value which is characteristic of a flowable medium inside the treatment chamber.

Particularly preferably, this measured value is selected from a group of measured values which includes pressure measured values, temperature measured values, measured values for a proportion of a sterilization agent, measured values for a concentration of the sterilization agent, air humidity values, and the like.

Particularly preferably, this sensor device is arranged inside the treatment chamber. Preferably, several sensor devices are provided and in particular these are also arranged in different positions within the treatment chamber.

In a further advantageous embodiment, the apparatus has at least one flow measuring device and particularly preferably several flow measuring devices. For example, the flow rate of the medium taken from the treatment chamber or the flow rate of the sterilization agent produced can be measured.

Particularly preferably, the apparatus comprises a plurality of flow lines, in particular a flow line for conveying the medium taken from the treatment chamber and also a flow line for conveying the medium (freshly produced by the generating device). Particularly preferably, flow measuring devices can also be provided in these flow lines.

In a further advantageous embodiment, the apparatus has at least one compressor device for compressing the flowable medium generated by the generating device. In a further advantageous embodiment, the apparatus has at least one compressor device for compressing the flowable medium removed from the treatment chamber.

Preferably, at least one compressor device is a blower or a side channel compressor. By using this compressor device, as mentioned above, gas can be taken directly from the treatment chamber and added to a supplied (fresh) gas stream, i.e., to the generated medium.

In this way, the flow velocity at the treatment elements, for example the above-mentioned holding devices for the plastic preforms or containers, can also be increased without the need for fresh gas or newly generated gas.

In a further preferred embodiment, the apparatus has an application device which is suitable and intended to apply at least part of the medium to be returned to the treatment chamber to at least one region of the plastic preforms and in particular to an external surface of the plastic preforms.

In the embodiments described above, an internal treatment of the plastic preforms was shown. Within the scope of this embodiment, it is proposed that the medium to be returned to the treatment chamber also be used for treating the external surfaces of the plastic preforms and/or plastic containers.

For these external treatments of the plastic preforms and/or plastic containers, nozzles are preferably used which apply medium to their external surfaces.

In this new development for external treatment, the approach was to carry out the treatment of the plastic preforms using additional nozzles. The gas mixture (in particular a mixture of H₂O₂ and air) which is already present in the clean room is sucked in, preferably compressed and/or condensed and supplied to the application devices and in particular nozzles.

Preferably, a compressor device and/or compression device is provided in this case as well, which compresses and/or condenses the mixture to be returned to the treatment chamber.

Preferably, the apparatus comprises nozzles arranged inside the treatment chamber, which are aligned and/or arranged such that they act on an external surface of the plastic containers or (in particular) plastic preforms.

Preferably, these application devices are arranged at least partially below the plastic containers and/or plastic preforms and advantageously apply medium to them from below.

Preferably, these application devices and/or nozzles are arranged in such a way that a targeted flow over the external surfaces occurs. These nozzles can complement existing nozzles.

Preferably, the medium is sucked in from the treatment chamber on an underside of the treatment chamber and/or from below (i.e., in a vertical direction from below). This suction on the lower side preferably improves the basic flow over the existing surfaces.

In this case, it is preferably possible to dispense with fans, which in comparable systems are arranged in the roof of the treatment chamber.

In a further preferred embodiment, the application devices and/or nozzles are arranged so as to be height-adjustable. Preferably, such a height adjustment can be carried out stepless. Such a height adjustment can preferably be carried out automated, preferably stepless, for example depending on the length of the plastic containers and/or plastic preforms.

In a further preferred embodiment, a blower may be provided to act on the external surfaces of the plastic preforms or plastic containers. A further flow measuring device can also be provided to allow for controlling this blower.

In a preferred embodiment, the blower is configured such that it can be supplied with the sterilization medium. In particular, this blower has components made of H2O2-resistant materials.

This procedure, in particular for the external treatment of the plastic preforms, has several advantages. First of all, no fresh gas mixture needs to be provided for the external treatment of the plastic preforms or plastic containers. Rather, existing escaping gas in the treatment chamber or clean room is utilized.

Furthermore, the described procedure can enable a reduction in the temperature and/or the concentration of the fresh gas. In addition, the procedure proposed here can also be implemented as a retrofit in existing systems.

Preferably, the application devices and/or nozzles are arranged in an area that is easily accessible through the setup access. This means that easy access to the application devices and/or nozzles should be possible or accessible from the setup access. Preferably, the application devices and/or nozzles are arranged at a distance from, in particular sufficiently spaced apart from, connection points to other, in particular adjacent, modules. This means that a sufficient distance must be maintained between the connection points in order to be able to continue to treat and/or handle the modules independently. Spaced apart, in particular sufficiently spaced apart, is regarded here as a distance that is usual for a person skilled in the art and which ensures ready handling of the modules, especially for installation/removal, maintenance and repair, as well as for manipulation.

Preferably, the application devices and/or nozzles are arranged both in an easily accessible area through the setup access and at a distance from the connection points to other modules.

The present invention is further directed to a method for treating and in particular sterilizing plastic preforms and/or containers (and in particular plastic containers), with a transport device which transports the plastic preforms and/or containers at least temporarily during their treatment.

Furthermore, a housing forming a treatment chamber is provided, within which the transport device is arranged and a generating device which generates a flowable treatment agent and in particular a flowable sterilization agent. Furthermore, a supply device is also provided which supplies the sterilization agent produced by the generating device to the treatment chamber.

Particularly preferably, a first discharge device is provided which removes a flowable medium enriched with the sterilizing agent from the treatment chamber, and also a return device is provided which supplies at least part of the flowable medium removed from the treatment chamber back to the same treatment chamber and/or returns it back to the treatment chamber.

Preferably, the apparatus comprises a processing device which is suitable and intended to process the medium taken from the treatment chamber, in particular before it is mixed with the freshly produced flowable medium. A heating apparatus for reheating the removed treatment gas, for example, is conceivable for this purpose.

It is therefore also proposed for the method that at least part of the sterilization medium is reused, and/or is removed from the treatment chamber and returned to it. However, particularly preferably, additionally produced sterilization agent is supplied to the treatment chamber. Particularly preferably, the quantities of the returned sterilization medium and the newly produced quantities of the sterilization medium and/or their proportions are controlled.

Particularly preferably, (freshly) produced sterilization medium is supplied to the treatment chamber in an amount that is greater than 100 kg/h, preferably greater than 200 kg/h, preferably greater than 300 kg/h and preferably greater than 400 kg/h.

Particularly preferably, the amount of (freshly) produced sterilization medium, i.e., newly produced sterilization agent, supplied to the treatment chamber is less than 1000 kg/h, preferably less than 800 kg/h, preferably less than 700 kg/h, preferably less than 600 kg/h.

Particularly preferably, the amount of sterilization agent removed from the treatment chamber and returned to the treatment chamber is more than 200 kg/h, preferably more than 400 kg/h, preferably more than 600 kg/h and preferably more than 800 kg/h. Preferably, the amount of sterilization agent and/or flowable medium removed from the treatment chamber and returned is less than 2000 kg/h, preferably less than 1800 kg/h, preferably less than 1600 kg/h and preferably less than 1400 kg/h.

Therefore, the proportion of sterilization medium removed from the treatment chamber and returned to the treatment chamber is particularly preferably higher than the proportion of newly produced sterilization medium.

Particularly preferably, the plastic preforms or containers are transported inside the treatment chamber at least in parts thereof on a circular transport path. Particularly preferably, plastic preforms are moved within the treatment chamber on at least two different transport planes, which differ in particular in the direction of the axis of rotation of the transport device and are particularly are preferably arranged one above the other in the direction of the axis of rotation, and are perpendicular to the axis of rotation. In this way, the efficiency of sterilization within the treatment chamber can be increased.

In a further preferred method, the plastic preforms or containers are transferred within the treatment chamber at least once and preferably at least twice from a first holding device to a second holding device.

In a further preferred method, the plastic preforms and/or (plastic) containers are transported within the treatment chamber at least once in different directions of rotation. Particularly preferably, the sterilization agent is applied to the plastic preforms or containers.

Particularly preferably, an internal surface of the plastic preforms or containers is also applied with the sterilization agent. This is preferably done by nozzles which are suitable for introducing sterilization agents into the interior of the plastic preforms.

In a further preferred method, the sterilization agent is distributed by a rotary distributor to movable nozzles located in particular on a rotatable carrier, and in particular, the above-mentioned transport carrier.

Particularly preferably, each of the above-mentioned holding devices is assigned exactly one or at least one application device.

In a further preferred method, a part of the flowable medium taken from the treatment chamber is mixed with the flowable medium produced by the generating device in a mixing device. It is particularly preferred that this mixing takes place outside the treatment chamber.

In a further preferred method, a mixing ratio between the medium produced by the generating device and the medium taken from the treatment chamber is controlled and/or regulated. This can be done, for example, by a pump device that can change the flow rate or by valves.

In a further preferred method, the plastic preforms and/or containers are supplied into the treatment chamber by an airlock device.

In a further preferred method, the plastic preforms and/or containers are removed from or discharged from the treatment chamber by an airlock device. Preferably, this airlock device reduces the escape of flowable medium from the treatment chamber.

In a further preferred method, the plastic preforms are heated before they are supplied into the treatment chamber.

Particularly preferably, the plastic preforms are formed into plastic containers after their sterilization by a forming device for forming plastic preforms. This is particularly preferably a sterile forming device, in particular a sterile blow molding machine and particularly preferably a sterile stretch blow molding machine.

In a further advantageous method, an application device is provided which applies at least part of the medium to be returned to the treatment chamber to at least one region of the plastic preforms and in particular to an external surface of the plastic preforms.

Preferably, the medium to be returned is accelerated by a blower. Preferably, the application device has at least one and preferably several nozzles. Preferably, the application device applies the medium to the plastic containers and/or plastic preforms and in particular the plastic preforms from below.

### BRIEF DESCRIPTION OF THE DRAWINGS

Further advantages and embodiments can be seen in the accompanying drawings, in which:

FIG. 1a shows a schematic representation of an apparatus according to the invention for internal sterilization;

FIG. 1b shows a schematic representation of an apparatus for external sterilization;

FIG. 1c shows a schematic representation of an apparatus for internal and external sterilization;

FIG. 2 shows a representation of the application of medium to plastic preforms.

FIG. 3 shows a representation of the return of the medium; and

FIG. 4 shows a representation of the application of medium to the external surface of plastic preforms.

### DETAILED DESCRIPTION OF THE INVENTION

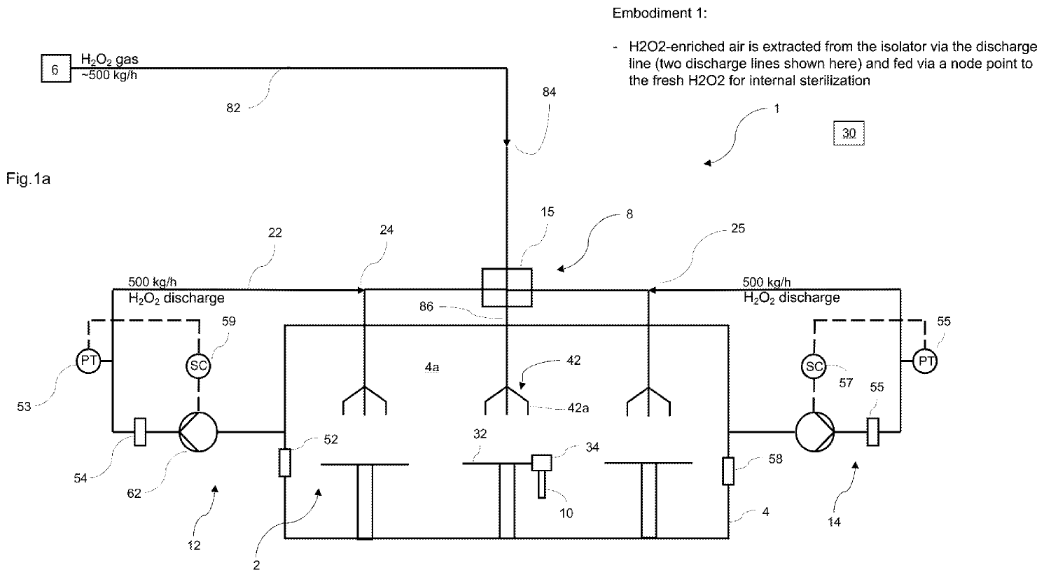

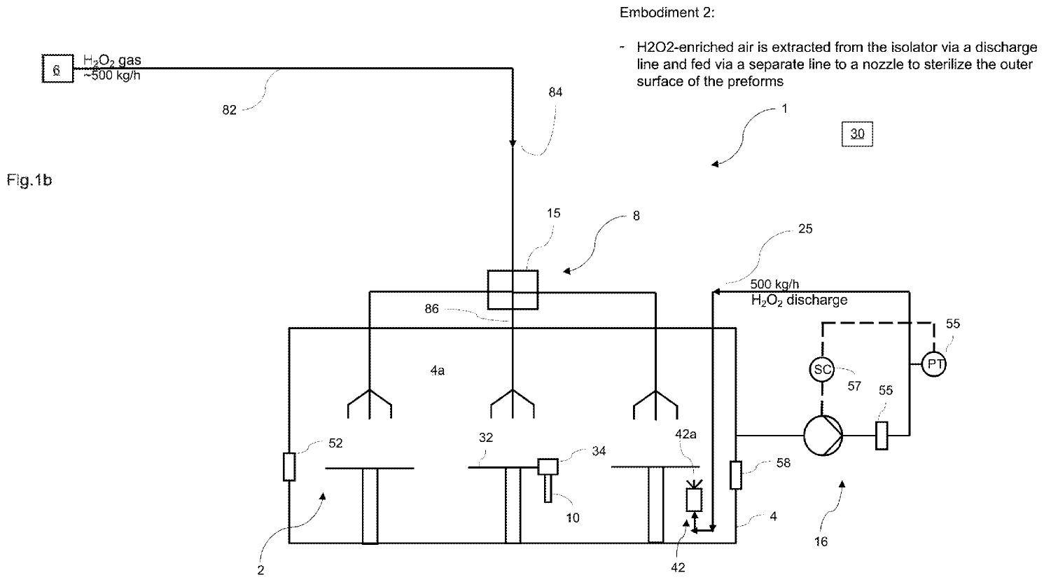

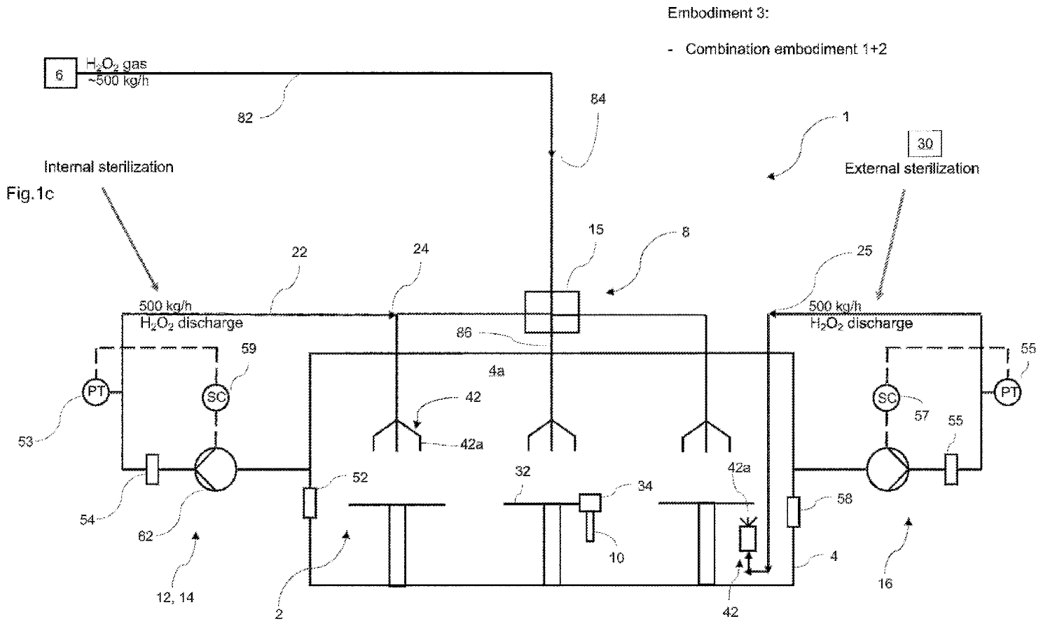

FIG. 1a shows an apparatus 1 according to the invention for treating plastic preforms and/or (plastic) containers. This has a preferably substantially closed housing 4, within which a treatment chamber 4a is formed for treating and in particular sterilizing, preferably for the internal sterilization of, plastic preforms 10.

The reference numeral 2 denotes in its entirety a transport device which here has three transport starwheels which are arranged one after the other within the treatment chamber 4a, so that each plastic preform 10 is transported (one after the other) by each of these transport units.

The reference numeral 6 denotes a generating device which is suitable and intended to generate a flowable sterilization agent, in particular H₂O₂ gas. Particularly preferably, this generating device is an evaporator, which is known per se from the prior art.

This sterilization medium can be supplied to the treatment chamber 4a via a connecting line 82 and a valve 84 as well as a further connecting line. Preferably, the valve 84 is a controllable valve and in particular a pneumatically or electrically operated valve.

The reference numeral 12 denotes a first discharge device which can discharge the medium located therein, i.e., in particular the gas enriched with sterilization agent, from the treatment chamber 4a. The reference numeral 62 denotes a compressor device, for example a pump for compressing the sterilization agent or gas taken from the treatment chamber.

The reference numeral 22 denotes a line device which supplies the flowable medium taken from the treatment chamber to the treatment chamber 4a. The reference numeral 24 denotes a valve device for controlling the gaseous medium to be returned to the treatment chamber. Preferably, the valve device 24 is also a controllable valve.

The reference numeral 14 denotes a second discharge device which is also suitable and intended to discharge the gas from the treatment chamber 4a. This second discharge device is preferably configured substantially in the same way as the first discharge device 12. The reference numeral 25 denotes a valve which fulfils the same function as the valve device 24 described above, i.e., the control of the flowable medium to be returned to the treatment chamber 4a.

The reference numeral 15 schematically indicates a mixing device in which the removed gas is mixed with the newly generated gas in order to then be supplied to the treatment chamber 4a via a line 86. Further mixing devices can also be provided in the other node points shown in FIG. 1.

The transport device 2 preferably has a rotatable transport carrier 32, on which a plurality of holding devices 34 for holding plastic preforms 10 are particularly preferably arranged. The reference numeral 42 denotes an application device which serves to apply the sterilization agent to the plastic preforms 10. This application device 42 has a plurality of application nozzles 42a, each of which introduces the sterilization agent into the plastic preforms.

Preferably, a plurality of sensor devices 52, 58 is provided in the treatment chamber, which can detect, for example, a gas pressure, a proportion of sterilization agent, or a temperature.

Furthermore, sensor devices 54, 56, for example in the form of flow measuring devices, are preferably provided outside the treatment chamber. The reference numerals 53, 55, 57, 59 represent further sensor devices. In the example shown, the designation “SC” stands for “Speed Control,” which is intended to clarify that, for example, a speed can be monitored. The monitored speed can, for example, be the speed of the pump, which could be controlled using a frequency converter, for example.

The reference numeral 30 denotes a control device which is used for controlling and regulating a mixing ratio of the gas to be introduced into the treatment chamber. Preferably, measurement signals and/or measured values of the individual sensor devices are also supplied to this control device 30, and the apparatus control is also carried out on the basis of these measured values.

Both the areas arranged schematically to the left and to the right of the substantially closed housing 4, with the treatment chamber 4a, are intended and suitable for treating and in particular sterilizing, preferably for internal sterilization of plastic preforms 10.

It is also possible to provide only one discharge device 12, 14.

FIG. 1b shows a similar schematic representation of an apparatus 1 for treating plastic preforms and/or (plastic) containers. This has a preferably substantially closed housing 4, within which a treatment chamber 4a is formed for treating and in particular sterilizing, preferably for external sterilization of plastic preforms 10.

The reference numerals with additional features can be found in the description of FIG. 1a. Elements provided with the same reference numerals identify corresponding elements as in FIG. 1a.

The reference numeral 16 denotes a discharge device which is suitable and intended to discharge the gas from the treatment chamber 4a. This discharge device 16 is preferably configured substantially in the same way as the discharge devices 12 and 14 in FIG. 1a.

An additional valve, corresponding to the reference numeral 25 in FIG. 1a, may also be provided therein.

The gas from the treatment chamber is led through the discharge device 16 in a separate line, preferably also via a valve or a node point, into the treatment chamber 4a, mixed with fresh gas (in particular enriched with sterilization agent).

This is preferably followed by an application device 42, which serves to apply the sterilization agent to the plastic preforms 10. This application device 42 preferably has a plurality of application nozzles 42a, or a single application nozzle 42a, each of which applies the sterilization agent to the plastic preforms.

The external surfaces of plastic preforms are preferably sterilized in this way.

At least two discharge devices 16 may also be provided.

FIG. 1c shows a further similar schematic representation of an apparatus 1 for treating plastic preforms and/or (plastic) containers. This has a preferably substantially closed housing 4, within which a treatment chamber 4a is formed for treating and in particular sterilizing, preferably for internal and external sterilization of plastic preforms 10.

A discharge device 12, 14 for preferred internal sterilization is shown schematically on the left, and a discharge device 16 for preferred external sterilization of plastic preforms 10 is shown schematically on the right.

At least two discharge devices 12, 14 for preferred internal sterilization and at least two discharge devices 16 for preferred external sterilization may also be provided.

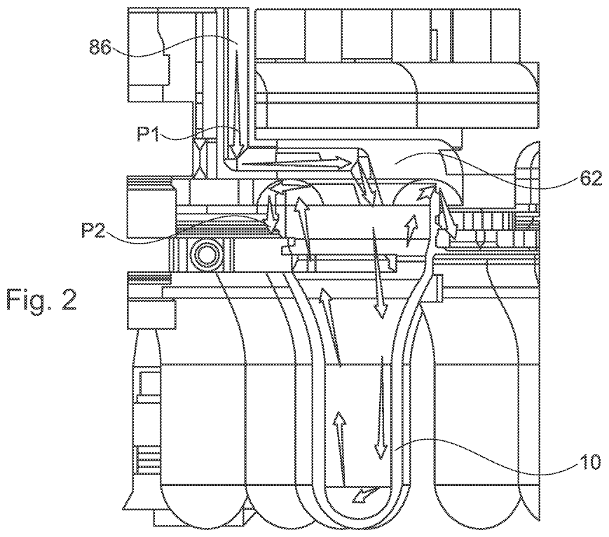

FIG. 2 is a detailed representation of the applying of a plastic preform 10—more precisely here, its internal surfaces. The medium returned to the treatment chamber is introduced into the plastic preform via line 86 (arrows P1). Above the plastic preforms there is preferably a distribution device 62 which ensures that medium emerging from the plastic preforms also reaches an external region of the mouth of the plastic preforms 10.

This distribution device 62 preferably has a circular cross section.

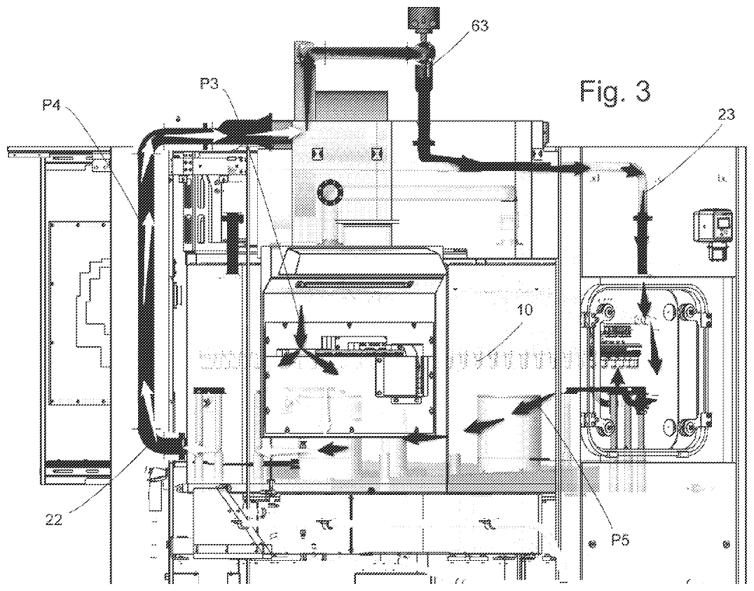

FIG. 3 shows a representation illustrating the return of the medium taken from the treatment chamber and its use for sterilizing the external surfaces.

The medium is discharged from the treatment chamber via a removal line 22. This removal line is preferably arranged in a lower region of the treatment chamber. The medium then flows (arrows P4 and P3) to a compressor device 63 and returns to the treatment chamber via a line (arrow P5).

The arrows P3 illustrate the application of medium to the respective interiors of the plastic preforms.

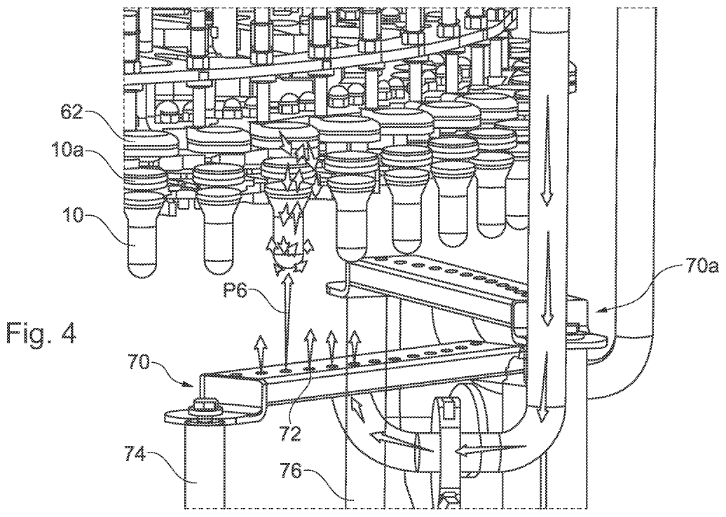

FIG. 4 shows the external application of medium to the individual plastic preforms. At least one application arrangement 70, 70a is provided within the treatment chamber. This application arrangement has a plurality of nozzles 72, which are preferably arranged one behind the other in a single row.

These nozzles preferably apply medium to the plastic preforms 10 from below.

The reference numerals 74 and 76 denote carriers for carrying the two application arrangements 70, 70a. The application arrangements are preferably adjustable in the vertical direction in order to enable adaptation to different geometries of the plastic preforms.

It is to be noted that all features which have been described with reference to the method are also disclosed in a corresponding manner for the apparatus, which means in particular that the corresponding apparatus has devices which are suitable and intended for carrying out the particular methods. Furthermore, features which were described with reference to the apparatus are also correspondingly applicable to the method or methods. This means that the methods are carried out using the corresponding apparatus features.

The applicant reserves the right to claim all features disclosed in the application documents as essential to the invention, provided that they are novel over the prior art individually or in combination. It is also pointed out that features which can be advantageous in themselves are also described in the individual figures. A person skilled in the art will immediately recognize that a particular feature described in a figure can be advantageous even without the adoption of further features from this figure. Furthermore, a person skilled in the art will recognize that advantages can also result from a combination of several features shown in individual or in different figures.

1. An apparatus for treating plastic preforms and/or containers, comprising a transport device which is configured for transporting the plastic preforms and/or containers during their treatment, a housing forming a treatment chamber within which the transport device is arranged, a generating device configured for generating a flowable sterilization agent, and a supply device which is configured for supplying the sterilization agent generated by the generating device to the treatment chamber,
wherein
the apparatus has a first discharge device which is suitable and configured for removing a flowable medium enriched with the sterilization agent from the treatment chamber, and a return device which is configured for returning at least part of the medium removed from the treatment chamber back to this treatment chamber and/or a further treatment chamber.

2. The apparatus according to claim 1,
wherein
the apparatus has at least one mixing device configured for mixing at least a part of the flowable medium taken from the treatment chamber with the flowable medium produced by the generating device.

3. The apparatus according to claim 1,
wherein
the apparatus has a control device which is configured for controlling a mixing ratio of the medium to be supplied to the treatment chamber and the medium removed from the treatment chamber.

4. The apparatus according to claim 1,
wherein
the apparatus has a second discharge device which is suitable and configured for removing a flowable medium enriched with the sterilization agent from the treatment chamber, wherein this second discharge device is preferably spaced apart from the first discharge device.

5. The apparatus according to claim 1,
characterized in that
the apparatus has at least one application device arranged within the treatment chamber and configured for applying the sterilization agent to the plastic preforms or containers transported by the transport device.

6. The apparatus according to claim 1,
wherein
the apparatus has at least one sensor device which is configured for detecting at least one measured value which is characteristic of the flowable medium located within the treatment chamber, wherein this measured value is selected from a group of measured values consisting of pressure measured values, temperature measured values, measured values for a proportion of a sterilization agent, measured values for a concentration of the sterilization agent and air humidity measured values.

7. The apparatus according to claim 1,
wherein
the apparatus has at least one flow measuring device.

8. The apparatus according to claim 1,
wherein
the apparatus has a compressor device configured for compressing the flowable medium generated by the generating device and/or the flowable medium removed from the treatment chamber.

9. The apparatus according to claim 1,
wherein
the apparatus has an application device which is configured for applying at least part of the medium to be returned to the treatment chamber to at least one area of the plastic preforms or plastic containers.

10. A method for treating plastic preforms and/or containers, with a transport device which transports the plastic preforms and/or containers at least temporarily during their treatment, with a housing forming a treatment chamber within which the transport device is arranged, with a generating device which generates a flowable sterilization agent, and with a supply device which supplies the sterilization agent generated by the generating device to the clean room,
wherein
a first discharge device is provided which removes a flowable medium enriched with the sterilization agent from the treatment chamber, and a return device which returns at least a part of the medium removed from the treatment chamber back into this treatment chamber.

11. The method according to claim 10,
wherein
the plastic preforms and/or containers are transported at least partially on a circular transport path within the treatment chamber and/or the plastic preforms and/or containers are transferred at least once within the treatment chamber from a first holding device to a second holding device.

12. The method according to claim 10,
wherein
at least a part of the flowable medium taken from the treatment chamber is mixed with the flowable medium produced by the generating device, wherein this mixing preferably takes place outside the treatment chamber.

13. The method according to claim 10,
wherein
a mixing ratio between the medium produced by the generating device and the medium taken from the treatment chamber is controlled and/or regulated.

14. The method according to claim 10,
wherein
the plastic preforms and/or containers are supplied into the treatment chamber by an airlock device and/or the plastic preforms and/or containers are removed from the treatment chamber by an airlock device and/or the plastic preforms and/or containers are heated before they are supplied into the treatment chamber.

15. The method according to claim 10,
wherein
an application device is provided which applies at least a part of the medium to be returned to the treatment chamber to at least one area of the plastic preforms.
